(19) United States
(12) Patent Application Publication
(10) Pub. No.: US 2021/0283074 A1
(43) Pub. Date: Sep. 16, 2021

(54) Polymeric Compositions for Intranasal Administration

(71) Applicant: Nasus Pharma Ltd., Tel Aviv (IL)

(72) Inventors: Tair Lapidot, Lapid (IL); Galia Temtsin-Krayz, Ashdod (IL); Carolina Abrutzky, Kfar Saba (IL); Dalia Megiddo, M.P. Judean Hills (IL)

(21) Appl. No.: 17/121,874

(22) Filed: Dec. 15, 2020

Related U.S. Application Data
(60) Provisional application No. 63/110,055, filed on Nov. 5, 2020.
(60) Provisional application No. 63/008,874, filed on Apr. 13, 2020.
(60) Provisional application No. 62/989,966, filed on Mar. 16, 2020.

(51) Int. Cl. A61K 31/14; C08K 5/092; C08K 5/098; C08K 5/19; A61K 9/00; A61K 47/12; A61K 47/22; A61K 31/192; A61K 31/055; A61K 31/4166; A61K 31/4178; A61K 31/197; A61K 33/00; A61K 31/19; A61K 31/225; A61P 31/12; A61K 9/06; A61P 31/16; A61K 9/16; A61M 15/08; A61M 15/00

(52) U.S. Cl. CPC A61K 31/14; A61M 2202/064; C08K 5/098; C08K 5/19; A61K 9/0043; A61K 47/12; A61K 47/22; A61K 31/192; A61K 31/055; A61K 31/4166; A61K 31/4178; A61K 31/197; A61K 33/00; A61K 31/19; A61K 31/225; A61P 31/12; A61K 9/06; A61P 31/16; A61K 9/1617; A61K 9/1652; A61M 15/08; A61M 15/009; C08K 5/092

(57) ABSTRACT

A composition in the form of dry powder for intranasal administration, having solid particles, each having a physiologically acceptable mucoadhesive polymer in combination with at least one functional additive such as pH adjusting agent, with at least about 90% of the particles having a size of about 25-300 microns, and various prophylactic and preventive uses thereof.

### CROSS-REFERENCE TO RELATED APPLICATIONS

The present application claims priority for U.S. Provisional Patent Application Ser. No. 62/989,966 filed on Mar. 16, 2020; from U.S. Provisional Patent Application Ser. No. 63/008,874 filed on Apr. 13, 2020; and from U.S. Provisional Patent Application Ser. No. 63/110,055 filed on Nov. 5, 2020, the contents of which are fully incorporated herein by reference.

### TECHNICAL FIELD

Disclosed are dry powder compositions for intranasal administration a polymeric agent capable of forming a bio-adhesive film upon intranasal administration methods for their preparation and uses thereof in medical and biological methods of prevention and treatment of various conditions.

### BACKGROUND

### Intranasal Drug Delivery

The respiratory tract is the most common route of viral entry, a consequence of the exposed mucosal surface and the resting ventilation rate of 6 liters of air per minute. The huge absorptive area of the human lung (140 square meters) also plays a role. Large numbers of foreign particles and aerosolized droplets—often containing bacteria, allergens and virions—are introduced into the respiratory tract incessantly. There are several defense mechanisms that protect the respiratory tract from infection by penetrating foreign bodies. For example, protection can be afforded by mechanical barriers—the respiratory tract is lined with a mucociliary layer comprising ciliated cells, mucus-secreting goblet cells, and subepithelial mucus-secreting glands. Foreign particles that enter the nasal cavity or upper respiratory tract are trapped in mucus and carried to the back of the throat, where they are swallowed. If particles reach the lower respiratory tract, they may also be trapped in mucus, which is then brought up and out of the lungs by ciliary action. The lowest reached alveoli are devoid of cilia; however, these gas-exchanging sacs are endowed with macrophages, whose function it is to ingest and destroy particles. Therefore, straightening of the mechanical barrier within the nasal cavity can provide improved protection of the upper respiratory tract from pathogens penetration.

Intranasal administration is known to be an efficient non-invasive method for systemic delivery of active agent, rapidly achieving effective relevant concentrations in the bloodstream, with no first-pass metabolism, and ease of administration.

The intranasal delivery of drugs utilizes devices of several types, such as nebulizers, pressurized devices, dry powder sprayers, and bi-directional nasal devices, for administration of single metered dose or multiple metered doses of an active agent.

Dry powders are used in intranasal drug delivery due to many advantages of using this dosage form including the improved stability, administration of larger doses and lack of microbial growth. The administration of intranasal powders may improve patient compliance, especially where the smell and taste of the delivered composition comprising excipients is unpleasant. Compared to drug solutions, the administration of powders can result in a prolonged contact with the nasal mucosa. Powder form is suitable for delivery of both small molecules and biologicals, especially peptides, hormones and antibodies.

WO2019/038756 describes a pharmaceutical composition in a form of dry powder for intranasal administration of various pharmaceutically active agents, the composition comprising two types of solid particles.

### SUMMARY OF THE INVENTION

The present disclosure relates to a composition in the form of dry powder for intranasal administration, said composition comprising first type particles, being essentially spherical solid particles comprising at least one physiologically acceptable mucoadhesive polymer and/or bio-adhesive polymer and/or gel-forming polymer in combination with at least one functional additive, wherein the size of at least about 90% of said first type particles is about 10-300 microns, wherein the size of at least 50% of the particles is about 30-100 microns and less than about 10% of said first type particles are of size of about 5-30, specifically less than 5 microns.

The presently disclosed composition can optionally further comprise second type particles being irregularly shaped solid particles comprising at least a pharmaceutically or physiologically acceptable inert carrier, wherein said second type particles are of a mean particle size greater than that of the said first type particles, preferably a mean particle size of from about 50 to about 200 microns.

In embodiments of the disclosed composition, the said composition can be substantially free of excipients other than said at least one functional additive comprised in said first type particles, and said second type particles can be only carrier or carrier with other excipient/s.

The mucoadhesive polymer and/or the bioadhesive polymer comprised in the presently disclosed composition can be a hydrophilic or amphiphilic gel-forming polymer. Specific examples of the mucoadhesive polymer and/or bioadhesive and/or gel-forming polymer comprised in the disclosed composition are, but not limited to hydroxypropylmethyl cellulose (HPMC, hypromellose), hydroxypropyl cellulose (HPC), sodium carboxymethyl cellulose (CMC), a natural gum such a xanthan gum, guar gum, gum acacia and gum tragacanth, starch, such as maize starch, potato starch, chitosan, algal sulfated polysaccharides, a hydrophilic methacrylic polymer, such a hydroxyethyl methacrylic polymer, or hydrophilic acrylic acid polymers such as Carbopol, polyethylene glycol, Poloxamer, polyvinyl alcohol and any co-polymer, grafted polymer and/or any mixture of at least two thereof. In more specific embodiments said mucoadhesive and/or bioadhesive polymer is hydroxypropyl-methyl cellulose (HPMC).

In embodiments of the compositions of the present disclosure the functional additive is a physiologically acceptable pH modifying agent. The pH modifying agent can an acidifying agent, specifically an acidifying agent which following intranasal administration and dissolution of the particles, provides the nasal cavity environment with a pH equal to or lower or higher than about 3.5, especially in the range of 3-4. Specific examples are, but not limited to, any one of L-pyroglutamic acid (PCA); ascorbic acid, citric acid, phytic acid, succinic acid, acetic acid, citric acid, hydrochloric acid, lactic acid, tartaric acid, malic acid, salts thereof hydrates, such as mono-, di- or tri-hydrates and anhydrates or monohydrate thereof.

In other specific embodiments of the composition of present disclosure, where present, the pH modifying agent is an alkalinizing agent, specifically an alkalinizing agent which following intranasal administration and dissolution of the particles, provides the nasal cavity environment with a pH higher than about 8. Specific examples are, but not limited to any one of sodium bicarbonate, sodium hydroxide, potassium hydroxide, monobasic potassium phosphate/sodium hydroxide solution providing pH 8; dibasic potassium phosphate; dibasic sodium phosphate; alkaline borate buffer comprised from boric acid and sodium hydroxide, potassium citrate, calcium carbonate, sodium lactate and calcium acetate and hydrates and anhydrates thereof.

In some embodiments of the composition of the present disclosure the functional additive is a physiologically acceptable agent which can be any one of antimicrobial agent, preserving agent, antiseptic agent, disinfectant, solubilizing agent, wetting agent or any mixture of at least two thereof. Specific examples are, but not limited to any one of benzalkonium chloride, benzoic acid, chlorocresol, diazolidinyl urea, imidurea, edetic acid and its salts, potassium sorbate, sorbic acid and its salts, benzethonium chloride or docusate sodium.

In embodiments of the presently disclosed subject matter, in the disclosed composition the ratio between said at least one muco-adhesive and/or bio-adhesive polymer and said at least one functional additive in said first type particles is between about 90.0-99.99% w/w.

Where present, said carrier can be any one of lactose monohydrate, lactose, a lactose functional analogue, or any mixture of at least two thereof. Alternatively, said carrier can be any one of dextrose, sorbitol, mannitol, maltitol and xylitol, a cellulose or cellulose derivative, or starch or starch derivative.

In all aspects and embodiments of the present disclosure the disclosed composition can further optionally comprise at least one pharmaceutical, alimentary or cosmetic scenting agent or fragrance. The said scenting agent or fragrance can be comprised in at least one of said first type particles, in said second type particles or in said dry powder.

The said scenting agent or fragrance can be, but is not limited to, at least one of a fruit-like scent, a flowery or plant scent, a food-like scent, a beverage-like scent or a perfume-like scent. A fruit-like scent can be, for example, apple, banana, citrus, berry, almond scent, or others. A flowery or plant scent can be for example a rose, lily, hibiscus, eucalyptus, mint, spearmint, rosemary, ginger, sage, pepper, lavender, cinnamon, vanilla scent or the like. A food-like scent can be, for example, candy scent, chocolate scent, honey scent or the like. A beverage-like scent can be, for example, coffee scent or the like.

The weight ratio between said first type particles and said second type particles, where present is from about 100:1 to about 1:10. Thus, in such compositions, the said first type particles can constitute from about 99.99% to about 10% of the composition.

In all aspects and embodiments of the present disclosure, following intranasal administration to a subject of a composition according to the present disclosure, homogeneous distribution and dissolution within the nasal cavity of the subject, the composition forms a protective polymeric film layer adhered to or a polymeric gel layer deposited on the nasal mucosa of said subject, which film layer or gel layer prevents at least in part foreign particulate bodies such as viral pathogens or allergens from accessing the nasal epithelium or penetrating the body of the subject. The particulate bodies can be of a mean size of from about 0.3 to about 3 microns.

In some embodiments the foreign particles can be the viral pathogens, bacteria or microbial pathogens.

In some embodiments, the said viral pathogen is a virus of any one of the Coronaviridae including SARS-CoV-2 (CoVID-19), Severe Acute Respiratory Syndrome virus (SARS-CoV) and Middle East Syndrome (MERS), Orthomyxoviridae, such as any of Influenza virus type A, Influenza virus type B or Influenza virus type C or any subtype or reassortant thereof including swine Influenza type A virus subtype H1N1 and avian Influenza type A virus subtype H5N1, Filoviridae, such as Marburg virus (MARV) and Ebola virus (EBOV), Flaviviridae such as Zika virus (ZIKV), West Nile virus (WNV), Dengue virus (DENV) and Yellow Fever virus (YFV) or Poxviridae families, and sub-families thereof. In specific embodiments, wherein said virus is SARS-CoV-2.

As can be seen in Example 8 below, a survey conducted during SARS-CoV-2 pandemic in Israel in a human population highly susceptible to COVID-19, showed that intranasal application of a composition according to the present invention comprising HPMC as the polymeric constituent and citric acid and/or sodium citrate as pH lowering agent (e.g. a composition of Example 1 below, also referred to as Taffix™), in addition to standard measure of wearing facial mask and social distancing), has practically prevented infection of users compared to non-users of the composition in the same group pf people, and has proven successful. Risk of infection was reduced by 4-fold compared to non-users in the same population (adhering to standard protection measures), and 10-fold lower than general population in same city, presenting a potential significant contribution in the fight against the current pandemic. Application of a composition according to the invention in addition to mask wearing and social distancing, provides significant additional protection to users for several hours. Furthermore, the present results suggest that a composition according to the present invention provided protection to people who used it as instructed, not only in community gathering/s but also at home, when not wearing a mask, even when an infected family member lived in the same household. Similar to SARS-CoV-2, the presently disclosed compositions and methods are expected to provide protection against infection by other viruses or viral fragments that may penetrate airways or body via the intranasal cavity.

In specific embodiments, the present disclosure provides a composition in the form of dry powder for intranasal administration to a subject in need, said composition comprising a first type of essentially spherical solid particles comprising HPMC in combination with a physiologically acceptable pH modifying agent, such as an acidifying agent or an alkalinizing agent, wherein the size of at least about 90% of said first type particles is about 10-300 microns, wherein the size of at least 50% of the particles is about 30-100 microns and less than about 10% of said first type particles are of size of about 5-30, specifically less than 5 microns.

In specific embodiments, the present disclosure provides a composition in the form of dry powder for intranasal administration to a subject in need, said composition comprising a first type of essentially spherical solid particles comprising HPMC in combination with a physiologically acceptable pH modifying agent, such as an acidifying agent or an alkalinizing agent, wherein the mean size of at least about 90% of said first type particles is about 40-150 microns, wherein the size of at least 50% of the particles are of a mean particle size of about 15-100 microns and less than about 10% of said first type particles are of a mean particle size of about 5-30, specifically less than 5 microns, providing a metered effective nominal dose of said HPMC, wherein following intranasal administration to said subject and dissolution in the nasal cavity of said subject, said composition forms a protective polymeric film layer or gel layer on the nasal mucosa.

Also in these specific embodiments, the said physiologically acceptable pH modifying agent can be an acidifying agent, for example, but not limited to any one of L-pyroglutamic acid (PCA); ascorbic acid, citric acid, phytic acid, succinic acid, acetic acid, citric acid, hydrochloric acid, lactic acid, tartaric acid, malic acid, salts thereof and hydrates and anhydrates thereof, and any mixture of at least two thereof, for example a mixture of citric acid and sodium citrate. Alternatively, said physiologically acceptable pH modifying agent can be an alkalinizing agent being any one of is any one of sodium bicarbonate, sodium hydroxide, potassium hydroxide, monobasic potassium phosphate/sodium hydroxide solution providing pH 8; dibasic potassium phosphate; dibasic sodium phosphate; alkaline borate buffer comprised from boric acid and sodium hydroxide, potassium citrate, calcium carbonate, sodium lactate and calcium acetate and hydrates and anhydrates thereof and any mixture of at least two thereof but not limited thereto. In these compositions the ratio between said at least one mucoadhesive and/or bioadhesive polymer and said at least one functional additive in said first type particles can be between about 90.0-99.99% w/w.

In other embodiments of the present disclosure the said functional additive can be any one of a physiologically acceptable antimicrobial agent, preserving agent, antiseptic agent, disinfectant, solubilizing agent, wetting agent or any mixture of at least two thereof. Specific examples are, but not limited to any one of benzalkonium chloride, ADBAC, benzoic acid, chlorocresol, diazolidinyl urea, imidurea, edetic acid and its salts, potassium sorbate, sorbic acid and its salts, benzethonium chloride or docusate, and any mixture of at least two thereof.

In a specific aspect of the composition of the present disclosure, the functional additive can be a mixture of said at least one pH modifying agent with said at least one physiologically acceptable antibacterial agent and/or a physiologically acceptable preserving agent (which may be also referred to herein as “a mixed functional additive”). A specific non-limiting example of a mixed functional additive is a mixture of at least one said pH modifying agent and at least one said antibacterial agent or preserving agent. In a specific embodiment such mixed functional additive comprises a mixture of citric acid and sodium citrate (as a pH acidic modifying agent) and benzalkonium chloride as an antibacterial/preserving agent, where the benzalkonium chloride may also serve as a surfactant.

In all said aspects and specific embodiments of the present disclosure, the disclosed composition can further optionally comprise second type particles being irregularly shaped solid particles comprising at least a physiologically acceptable carrier, wherein said second type particles are of a mean particle size greater than that of the said first type particles, preferably a mean particle size of from about 50 to about 200 microns. The carrier can be any one of lactose monohydrate, lactose, a lactose functional analogue, or any mixture of at least two thereof. Alternatively, carrier can be any one of dextrose, sorbitol, mannitol, maltitol and xylitol, a cellulose or cellulose derivative, or starch or starch derivative. The weight ratio between said first type particles and said second type particles, where present is from about 100:1 to about 1:10. Thus, in such compositions, the said first type particles can constitute from about 99.99% to about 10% of the composition. The particulate foreign bodies can be of a mean size of from about 0.3 to about 20 microns.

In all aspects and embodiments of the present disclosure the viral pathogen can be a virus of any one of the Coronaviridae including SARS-CoV-2, Severe Acute Respiratory Syndrome virus (SARS-CoV) and Middle East Respiratory Syndrome (MERS), Orthomyxoviridae, such as any of Influenza virus type A, Influenza virus type B or Influenza virus type C or any subtype or reassortant thereof including swine Influenza type A virus subtype H1N1 and avian Influenza type A virus subtype H5N1, Filoviridae, such as Marburg virus (MARV) and Ebola virus (EBOV), Flaviviridae such as Zika virus (ZIKV), West Nile virus (WNV), Dengue virus (DENV) and Yellow Fever virus (YFV) or Poxviridae families, and sub-families thereof. In specific embodiments said virus is SARS-CoV-2, causing CoVID-19. A “virus” as used herein is to be taken to mean also any infectious fragments and particles thereof.

The disclosed compositions can be used in preventing at least part foreign particulate bodies such as viral pathogens or allergens from accessing nasal epithelium or penetrating the body of the subject.

The effective dose of HPMC in compositions according to the invention can be from about 0.01 mg to 20 mg.

The disclosed compositions can be designed for from one to six daily intranasal administrations, for example once, twice, three, four, five or six times daily, for example 1-2, 1-3, 1-4, 1-5, 1-6, 2-3, 2-4, 2-5, 2-6, 3-4, 3-5 or 3-6 daily. Frequency of application is generally to be provided in instructions-for-use of the composition, taking into account environmental conditions such as crowding and density of people, risk of exposure to virus carriers, time of exposure, and the like.

Any of the disclosed compositions can be contained in an airtight container or inhaler suitable for intranasal administration of a powder, for personal use. For example, the composition can be contained in a container for personal use that provides said composition at a metered dose and it suitable for multiple administrations, specifically wherein said container is of a volume of from about 10 ml to about 500 ml. The said scenting agent or fragrance can be added to the container together with the dry powder, in addition to any scent comprised in the powder or there instead.

Still further, the present disclosure provides a method for preventing or reducing infection by a viral pathogen, particularly respiratory viral infection, comprising intranasally administering to a subject in need an effective amount of a composition as disclosed and claimed herein. Administration can be by the subject himself, or by an assisting person or by a member of medical staff. Specifically, the composition is self-administered. Administration can be repeated administration of from one time to six times daily, over a period of from 1 to 30 or more days. The method of treatment or prevention can be effective in preventing or reducing acuteness of cytokine storm in a patient, induced by said viral pathogen.

### BRIEF DESCRIPTION OF THE DRAWINGS

In order to better understand the subject matter that is disclosed herein and to exemplify how it can be carried out in practice, embodiments will now be described, by way of non-limiting example only, with reference to the accompanying drawings, in which:

FIG. 1 shows the particle distribution curves of raw HPMC and microspheres of the present invention prepared in Example 1a.

FIG. 2 shows Scanning Electron Microscope image of the microspheres of the present invention prepared in Example 1a

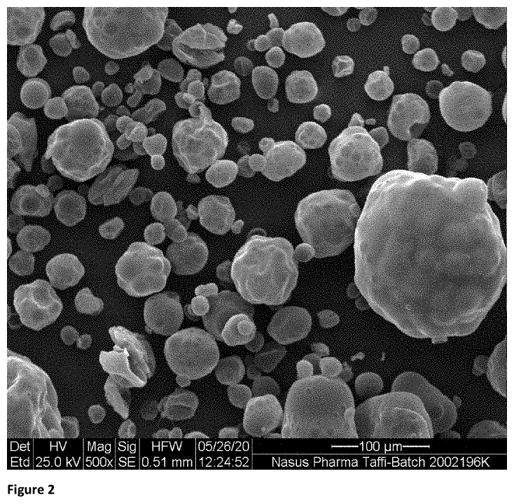

FIG. 3 shows protection of cells against viral infection by a composition according to present disclosure.

FIG. 4 shows protection of cell viability following exposure to H1N1 virus by a composition according to the present disclosure.

FIG. 5 shows rate confirmed SARS-CoV-2-infected persons in different populations, as described in Example 4.

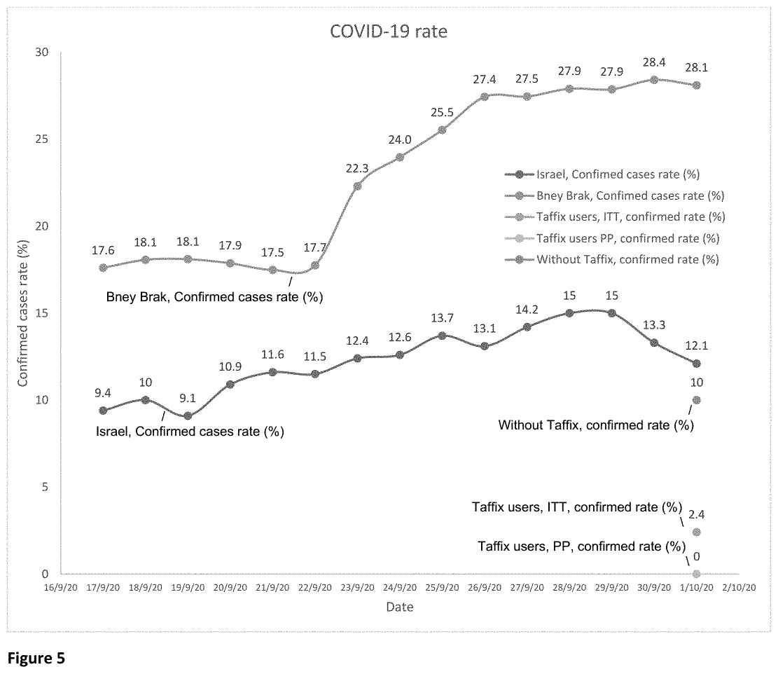

### DESCRIPTION OF EMBODIMENTS

Disclosed herein are novel compositions in the form of dry powder, for intranasal administration of a physiologically acceptable muco-adhesive and/or bio-adhesive polymer. Generally, a formulation according to the present disclosure comprises at least one type of solid particles (also referred to herein as first type particles). These are essentially spherical solid particles, comprising at least one muco-adhesive and/or bio-adhesive polymer in combination with a pH modifying agent and optionally a biocidal/bactericidal agent, wherein the particles size distribution is such that the size of at least about 90% of said first type particles is about 10-300, 15-300, 20-300 or 25-300 microns, wherein the size of at least 50% of the particles about 30-100, 35-100 or 40-100 microns and less than about 10%, such as less than 9.5%, 9%, 8.5%, 8%, 7.5%. 7&, 6.5% or 6% or less of said first type particles are of size of about 5-30 microns, such as less than 30, 25, 20, 15, 10 or 5 microns.

Optionally, the disclosed compositions comprise a second type of solid particles, specifically irregularly shaped solid particles comprising an essentially inert physiologically acceptable component, which serves as carrier or diluent. Where present, the mean size of the second type particles is greater than that of the first size particles.

Upon intranasal administration to a subject of a composition according to the present invention, the polymeric component of the first type particles is solubilized and forms a muco-adhesive film layer and/or gel layer adhered to and/or deposited on the nasal cavity mucosa. The film and/or gel layer prevent at least in part foreign particulates bodies such as viral pathogens and allergens from reaching the nasal epithelium and from penetrating the body of the subject. Moreover, upon solubilization of the first type particles, the pH modifying agent modifies the pH of the environment adjacent to said film or gel to below about 3.5, when an acidifying agent is used, or to above about 8, when an alkalinizing agent is used. Viral pathogens are known to be destroyed at pH of under 3.5 or above 8. Thus, any viral pathogens reaching the nasal cavity are not only blocked from reached the subjects' nasal epithelium and penetrating the body, they are also destroyed in situ, once in contact with the acidic or alkaline environment created in the nasal cavity.

Compositions for intranasal administration in dry powder form are usually produced by milling techniques. As a result, their particle size distribution is broad and the particles are usually non-spherical and non-uniform. The presence of particles of a mean size of less than 5 microns (μm) should however be avoided. Such very small particles may reach the lung mucosa by nasal spraying or by inhaling, which is unacceptable for intranasal administration from safety point of view. Therefore, the size distribution in embodiments of the presently disclosed compositions is such that about 40-90% said polymer-containing particles are of a mean particle size of about 40-150 microns, at least 50% of the particles are of a mean particle size of about 15-100 microns and less than about 10% of the particles are of a mean particle size of about 5-30. The presence of less than 10% of particles having a mean diameter of less than 5 microns, renders their use in nasal spraying beneficial for the intranasal administration.

In all embodiments of all aspects of the present disclosure, the physiologically acceptable polymeric component is mucoadhesive polymer and/or is bioadhesive polymer and/or is a gel-forming polymer, which can be a hydrophilic or amphiphilic gel-forming polymer, for example but not limited to any one of hydroxypropylmethyl cellulose (HPMC), hydroxypropyl cellulose (HPC), sodium carboxymethyl cellulose (CMC), a natural gum such a xanthan gum, guar gum, gum acacia and gum tragacanth, starch, such as maize starch, potato starch, chitosan, algal sulfated polysaccharides, a hydrophilic methacrylic polymer, such a hydroxyethyl methacrylic polymer, or hydrophilic acrylic acid polymers such as Carbopol, polyethylene glycol, Poloxamer, polyvinyl alcohol and any co-polymer, grafted polymer or and any mixture of at least two thereof.

The said functional additive can be, but is not limited to, a pH modifying agent or an antibacterial/preserving agent or a mixture of at least two thereof as described above.

The inert carrier, where present, can be any of the carriers listed above.

In all aspects and embodiments of the present disclosure the present composition can substantially free of excipients other than the at least one functional additive comprised in said first type particles and the optional inert carrier comprised in said second type particles.

The composition according to the present disclosure can be contained in suitable containers as described above that are user friendly and designed to enable systemic delivery of small and accurately metered doses of intranasal powder compositions by patients themselves or caregivers who are not healthcare professionals or medically trained. Such self-administration is also referred to herein as personal use.

Thus, in a further aspect the present disclosure relates to a container comprising a composition according to the present disclosure, that can provide predetermined multiple doses of the composition. The container can be designed for personal use by the subject in need, namely self administration, or by another person.

The disclosed composition can be prepared by a modified spray drying method, as described for example in WO2019/038756, fully incorporated herein by reference, and in the following Examples. Thus, to prepare particles of said first type, all constituents, specifically the polymeric component, the pH adjusting agent and the bactericidal agent where present, are first mixed with a solvent, and the solution is then subject to spray-drying to give a free-flowing powder.

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The terminology used herein is for describing particular embodiments only and is not intended to be limiting of the invention. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

### Definitions

The terms “bio-adhesive polymer”, “muco-adhesive polymer” and “gel-forming polymer” and the like used herein interchangeably, refer to a polymer which following intranasal administration to a subject and dissolution, is capable of forming a polymeric film and/or polymeric gel adheres to and/or is deposited on nasal cavity mucosa for at least 1-6 hours, and can also refer to a mixture of at least two such polymers.

The terms “formulation”, and “composition” may be used herein interchangeably, and are to be taken to mean a formulation comprising a physiologically acceptable bio-adhesive or muco-adhesive polymer as defined above, for use in therapeutic and preventive methods of treatment.

The terms “inert” or “inactive” or “inactive ingredient” or “inert ingredient”, as used interchangeably herein refer to components of the composition, or used in the preparation thereof, that do not instantly react with the active ingredient or adversely affect its properties, or cause any biological effect upon administration to a subject when administered at reasonable amounts to a subject. The general examples of these components are described in “The Handbook of Pharmaceutical Excipients”, 4^{th} Edition, by Rowe, Sheskey and Weller, Pharmaceutical press, 2003. Additional exemplary list is Inactive Ingredients Guide of the Food and Drug Administration, USA.

“Carrier” and “diluent” are used herein interchangeably and refer to an inert ingredient added to the composition.

“Scent”, “scenting agent” and “fragrance” as used interchangeably herein refer to any chemical agent that provides fragrance to the nasal cavity or improves the olfactory properties of the composition upon administration and dissolution in the nasal cavity, and is pharmaceutically/physiologically suitable for nasal administration.

A “patient” or “subject” that may be administered with the composition according to the presently disclosed subject matter is generally a human in need for prevention and/or treatment of infections or disorders induced by foreign particulate bodies, for example viral pathogens or allergens.

“pH adjusting agent”, “pH modifying agent” “buffering agent” and “buffer” as used herein interchangeably are to be taken to mean any chemical agent that affects the pH of its immediate environment/vicinity.

The terms “antibacterial/bactericidal agents” and “biocidal/bactericidal agent” are used herein interchangeably. Such agents can also be “preserving agents”.

The term a composition or substance “substantially free of excipients” is to be taken to mean that it does contain more than 5% by weight of such excipient/s.

The terms “treat”, or forms thereof, and the term “alleviate” and the like are to be taken to mean at least partially ameliorate or cure or totally eliminate the patient's condition as defined herein. The term “prevent” as used herein or forms thereof are to be taken to mean preventing or arresting any penetration via the nasal cavity into to the airways or body of a treated subject of harmful foreign particulate bodies such as viruses and allergens.

The term “suitable” as used herein is to be taken to mean having the properties that enable providing the defined result.

The terms “physiologically acceptable” and “physiologically compatible” as used herein and variations thereof are to be taken to mean a substance that does not interfere and does not have any adverse effect on the body of a treated subject and its functioning. This term may be interchangeably used with “pharmaceutically acceptable” and “pharmaceutically compatible”.

The terms “size” and “mean size” may be used herein interchangeably, and generally refer to a mean size or mean size range of a recited population or sub-population of particles comprised in the disclosed powder composition.

“About” as used herein generally refers to approximate values. When referred to a dose of drug, or size of particles and the like, “about” should be understood as including the range of a value ±15%. When referred to other values, the term should be understood as including the range of a value ±15%, for example ±15%, ±12%, ±10%, ±8%, ±5%, ±2% or ±1%. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

As used in the specification and claims, the forms “a”, “an” and “the” include singular as well as plural references unless the context clearly dictates.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The presently disclosed subject matter is further illustrated by the following examples, which are illustrative only and are not to be construed as limiting the scope of the invention. Variations and equivalents of these examples will be apparent to those skilled in the art in light of the present disclosure, the drawings and the claims herein.

It is appreciated that certain features of the presently disclosed subject matter which are, for clarity, described in the context of separate embodiments, can also be provided in combination in a single embodiment. Conversely, various features of the presently disclosed subject matter, which are, for brevity, described in the context of a single embodiment, can also be provided separately or in any suitable sub-combination.

Although the presently disclosed subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as relevant prior art to the presently disclosed subject matter.

### DESCRIPTION OF NON-LIMITING EXAMPLES

### Materials

### Polymer/s

Hydroxypropylmethyl cellulose (HPMC), hydroxypropyl cellulose (HPC), sodium carboxymethyl cellulose (CMC) from Dow chemicals, a natural gum such a xanthan gum, guar gum, gum acacia and gum tragacanth, starch, such as maize starch, potato starch, chitosan from Merck-Sigma-Aldrich a hydrophilic methacrylic polymer, such a hydroxyethyl methacrylic polymer, or hydrophilic acrylic acid polymers such as Carbopol from Lubrizol, polyethylene glycol, Poloxamer, polyvinyl alcohol from BASF.

### pH Modifying Agents

L-pyroglutamic acid (PCA); ascorbic acid, citric acid, phytic acid, succinic acid, acetic acid, citric acid, hydrochloric acid, lactic acid, tartaric acid, malic acid, salts thereof hydrates and anhydrates or monohydrate from Merck-Sigma-Aldrich.

### Antibacterial/Surfactant Agents

Benzoic acid, chlorocresol, diazolidinyl urea, imidurea, edetic acid and its salts, potassium sorbate, sorbic acid and its salts, benzethonium chloride from Merck-Sigma-Aldrich.

### Scenting/Fragrance Agent

Menthol and vanillin were purchased from Merck-Sigma-Aldrich.

### Example 1: Preparation of a Composition of HPMC with Acid

To 100 ml of 10% HPMC solution (Benecel E3) 1.85 gram of 0.1M citric acid solution were added providing pH 3.62. This viscous solution was spray dried at inlet temperature 120° C. (outlet 80-82° C.); pump 10; aspirator 100%; air flow 30 on scale. The free-flowing powder was obtained.

### Example 1a: Preparation of a Composition of HPMC with Acid and Benzalkonium Chloride

To 100 ml of 9% HPMC solution were added 0.7 g citric acid, 0.3 g sodium citrate and 0.01 g of Benzalkonium chloride, providing pH 3.7. The resulting viscous solution was spray-dried at inlet temperature 120° C. (outlet 80-82° C.); pump 10; aspirator 100%; air flow 30 on scale. A free-flowing powder was obtained.

### Example 1b: Preparation of a Composition of HPMC with Acid, Benzalkonium Chloride and Menthol

To 100 ml of 9% HPMC solution were added 0.7 g citric acid, 0.3 g sodium citrate, 0.01 g of Benzalkonium chloride and 0.2 g of menthol, providing pH 3.7. The resulting viscous solution was spray-dried at inlet temperature 120° C. (outlet 80-82° C.); pump 10; aspirator 100%; air flow 30 on scale. A free-flowing powder was obtained.

### Example 2: Preparation of Composition of HPMC with Base

To 100 ml of 10% HPMC solution (Benecel E3) 0.21 gram of 0.1M Sodium hydroxide solution was added providing pH 8.97. This viscous solution was spray dried at inlet temperature 120° C. (outlet 80-82° C.); pump 10; aspirator 100%; air flow 30 on scale. The free-flowing powder was obtained.

### Example 2a: Preparation of a Composition of HPMC with Base and Benzalkonium Chloride

To 100 ml of 10% HPMC solution (Benecel E3) were added 0.21 g 0.1M Sodium hydroxide solution and 0.01 g benzalkonium chloride, providing pH 8.97. The viscous solution obtained was spray-dried at inlet temperature 120° C. (outlet 80-82° C.); pump 10; aspirator 100%; air flow 30 on scale. A free-flowing powder was obtained.

### Example 2b: Preparation of a Composition of HPMC with Base, Benzalkonium Chloride and Menthol

To 100 ml of 10% HPMC solution (Benecel E3) were added 0.21 g 0.1M Sodium hydroxide solution, 0.01 g benzalkonium chloride and 0.2 g of menthol providing pH 8.97. The viscous solution obtained was spray-dried at inlet temperature 120° C. (outlet 80-82° C.); pump 10; aspirator 100%; air flow 30 on scale. A free-flowing powder was obtained.

### Example 3: Preparation of a Composition of HPMC with Salt

To 200 ml of 3% HPMC and 1% NaCl solution (Benecel E3) pH 5.5. This viscous solution was spray dried at inlet temperature 120° C. (outlet 80-82° C.); pump 15; aspirator 100%; air flow 30 on scale. The free-flowing powder was obtained.

### Example 4: Particle Size Distribution

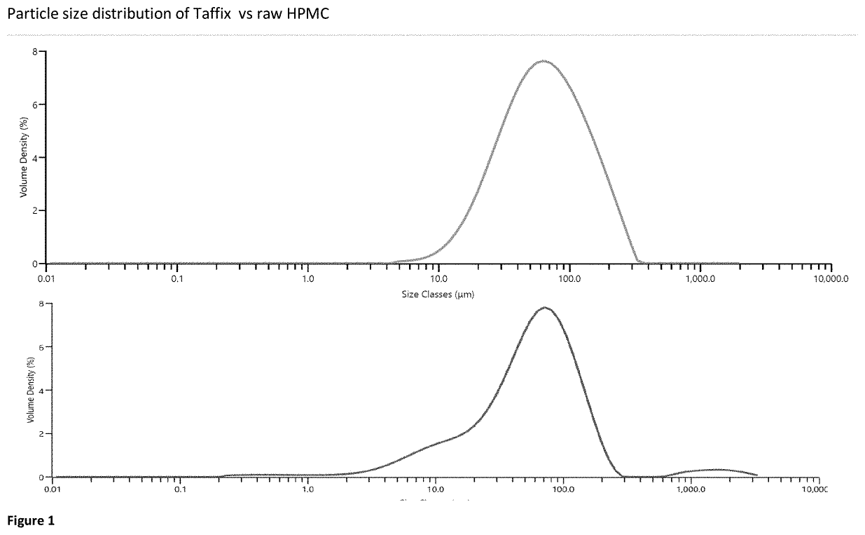

The obtained composition of Example 3 and starting HPMC material were subjected to particle size analysis using a Malvern Laser Diffraction instrument. As shown in FIG. 1, the following particle size distributions were obtained:
i. Benecel 3E (Ashland)—HPMC unprocessed: D₁₀—11.2 microns; D₅₀—58.3 microns; D₉₀—144 microns and D₁₀₀—3470 microns;
ii. Example 1a (Nasus) D₁₀—22.8 microns; D₅₀—62.6 microns; D₉₀—163 microns and D₁₀₀—345 microns

The particle size distribution of spray dried micro spheres of the present inventions are much more uniform, have better spray ability and solubility. In addition they meet safety requirements for Nasal powders. [Nasal Spray and Inhalation Solution, Suspension, and Spray Drug Products—Chemistry, Manufacturing, and Controls Documentation; Guidance for Industry; CDER, July 2002].

### Example 5: Protection of Cells Against GFP Lentivirus Infection

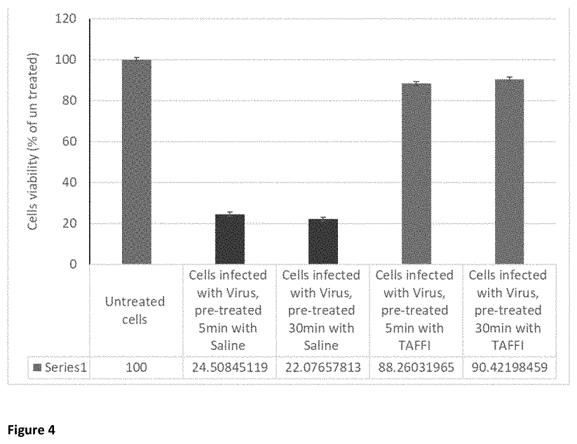

MDCK cells were incubated with polymeric compositions prepared according to the present inventions with pH=3.5 (according to Example 1a, TAFFIX in FIG. 4)) and pH=7.9 (according to Example 2), and the protective effect of gel (mechanical) and low pH (chemical) against viral infection was tested using a fluorescent GFP lentivirus.

As shown, cells that were not protected by a composition according to the invention were significantly infected with the virus, while cells protected with a gel according to the invention were considerably less infected, showing the protective effect of the gel against viral penetration.

### Example 6: Protection of Cell's Viability Upon Exposure to N1H1 Influenza Virus

MDCK cells pre-treated with a composition as prepared in Example 1 were exposed to N1H1 Influenza virus for 5 and 30 minutes, and the protective effect of the composition was tested in comparison to cells pretreated with saline solution. Cell viability was measured using a cell proliferation assay kit (XTT based).

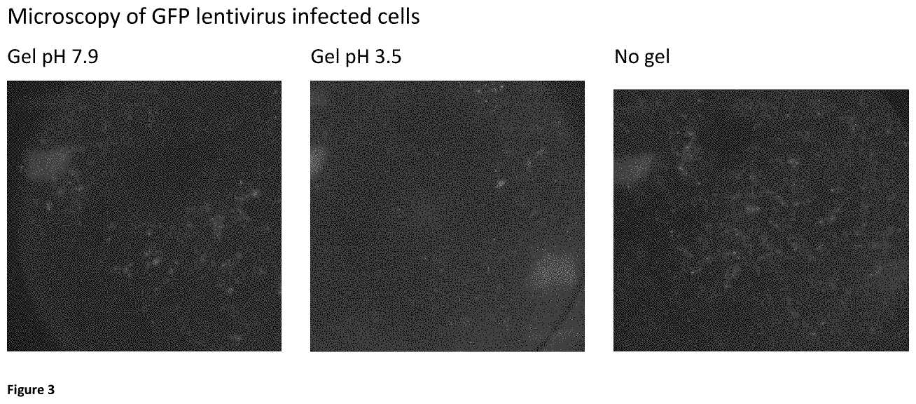

The results are shown in the following FIG. 3.

As shown in FIG. 3, pretreatment of the viruses with saline for 5 minutes showed reduced cells viability to 27% and pretreatment of viruses with HMPC alone (pH-6.8) reduced cell viability to 37%.

Remarkably, pretreatment with the composition of Example 1 (TAFFIX in the Figure) protected 88% of cells after 5 minutes pretreatment, and 90% after 30 minutes pretreatment, attesting to a fundamental role of acidity in disabling aggressive respiratory viruses.

### Example 7: Protection of Cell's Viability Upon Exposure SARS-CoV-2

The composition according to Example 1 was tested was to test establish that it can form a protective barrier against SARS-CoV-2. A gel of the composition was pre-formed on a 40 μm nylon filter, and then seeded with 10,000 PFUs of virus. An untreated filter, seeded with the same amount of virus, was used as an untreated control. After a 10-minute incubation the bottoms of the filters were washed with culture medium and then tested for live viruses by plaque assay and for viral RNA using qRT-PCR. The composition of Example 1 reduced the amount of live viruses by more than 99%, and in most experiments no virus was detected at all or the amount of virus present was below the limit of detection of the assay in the undiluted flow through. Using qRT-PCR techniques, treatment with the composition of Example 1 reduced the amount of viral RNA by more than 4 logs.

### Example 8: Protection Against SARS-CoV-2 Infection—Survey in Humans

A composition of Example 1 (referred to below by Taffix™) was used in the survey described below.

Materials and Methods

Spray containers for intranasal administration of the powder Taffix composition were used, each for personal use by each participant of the survey. The container provides the composition at a metered dose and it suitable for multiple administrations. In the current survey, the volume of the container was 20 ml. and amount of the single administered Taffix powder was 3-5 mg in each nostril. The powder is administered into the nose of the user by insufflation every 5 hours by gentle press of the plastic container (bottle with dropper) and directing the dropper toward one, then other nostril.

Population:

During the high holiday season in Israel a prospective user survey in an ultraorthodox community the city of Bnei Brak (BB), Israel. “Real-life” effect of Taffix in preventing infection following large social gathering in a closed environment was surveyed in BB around the Rosh Hashana (Jewish New Year) holiday, such gathering being a potential “super-spread” event.

Collaboration was with a medium sized synagogue community of some 250 members. Following preliminary presentation of information and notification, members of the community expressed their interest in using Taffix throughout Rosh Hashana prayers and over the following two weeks. Each member could collect a Taffix container (for intranasal administration at metered doses, which is suitable for multiple administrations, as described herein) at the synagogue and received instructions for proper usage of the device. Members also committed to use Taffix whenever encountering a large social interaction and re-apply Taffix every 5 hours when still in a crowded area, for the two weeks following the Holiday. Cooperation of the community members strongly supported and encouraged by the spiritual leadership of the community. Weekly reminders to all participants were sent directly through the community email system and close attention and monitoring was carried out to substantiate the number of new cases in the community. It was explained to participants that use of Taffix offers an extra layer of protection in addition to, not in replacement of mandatory use of masks and adherence to social distancing.

Taffix containers were collected by participants one day before the Holiday. All in all 113 containers of Taffix were collected by community members.

By day 14 after the Rosh Hashana holiday, members of the community were all contacted and were asked to report whether they have used Taffix, how often and under what circumstances they used it, and whether there were new cases of COVID-19 (SARS-CoV-2 infection) since the Holiday. Information about the total confirmed new COVID-19 cases in the remainder of the community was also collected and confirmed.

Results

243 members of the Synagogue participated in the two days holidays prayers (at least 7 hours spent in the synagogue each day in a closed room).

113 containers were collected by the synagogue members one day before the holiday. Eighty-three (83) members, men, women and children above the age of 12 years, actually used Taffix, out of which 81 used it regularly as instructed (Per-Protocol, PP participants) before entering a populated area and every 5 hours, two (Intent-to Treat, ITT participants) used it improperly “once or twice” throughout the 14 days period. There were no reports of side effects and most users commented on ease of use and had no problem in adapting the use of Taffix to their daily routine.

One hundred and sixty (160) members of the either did not collect the Taffix at all or collected it and did not use it at all, not even once.

All 81 members who used Taffix regularly according to the instruction for use were not infected at all during the study period of 14 days following Rosh Hashana.

18 members of the community were confirmed as new COVID 19 infected patients in the 14 days following the Holiday, of which 16 did not collect or use Taffix, two collected Taffix and used it incorrectly, that being only once or twice throughout the whole two weeks period ITT). One of them was diagnosed only 2 days after Rosh Hashana.

In two families, one family member who did not use Taffix was confirmed positive while the other family members sharing the same household who did use Taffix (PP) were not infected during the 14 days of follow-up. The Rabbi, who used Taffix regularly was not infected, while a man who was sitting directly next to him during the 7 hours of prayer services and did not use Taffix was one of those infected. To be clear, the Rabbi (Taffix user) did not get infected by the infected (non-Taffix user) congregant, who sat next to him for hours.

Of note in this time period the number of new cases in the city of Bnei Brak increased by 1.6 folds as positivity rates went from 17.6% to 28.4%. In this connection it is noted that the tested population generally adhere to wearing masks and social distancing, hence the difference between the members not using Taffix and the general population of Bnei Brak.

Statistical Analysis:

Statistical Methods

Results analysis was performed first on the ITT (Intent-to-Treat) population data (members who occasionally used Taffix) and then on the PP (Per-Protocol) population (members who used Taffix regularly according to instructions).

The Fisher's exact test for the comparison of two proportions (from independent samples), expressed as a percentage, was applied to compare the contagion rate between Taffix users and non-users.

Fisher's exact test is used to calculate an exact P-value for a 2×2 frequency table with small number of expected frequencies.

All tests are two-tailed, and a p-value of 5% or less is considered statistically significant.

The data was analyzed using the SAS® version 9.4 (SAS Institute, Cary N.C.).

Statistical Results

In the ITT population, 2.4% of the Taffix users (2/83) and 10% of the Taffix non users (16/160) were infected, thus a difference of 7.6%.

| | |
|---|---|
| Difference | 7.6% |
| 95% CI | 0.6% to 13.6% |
| Significance level | P = 0.037 |

In the PP population, 0% of the Taffix users (0/81) and 10% of the Taffix non users (16/160) were infected, thus a difference of 10%.

| | |
|---|---|
| Difference | 10.0% |
| 95% CI | 4.1% to 15.6% |
| Significance level | P = 0.002 |

For both the populations, the contagion rate for the Taffix users was significantly lower than for Taffix non-users.

The difference in levels of infected members between the tested non-users population (10%), total Bnei Brak population (28.1%) and total Israeli population (12.1%) during the test period can be attributed to adherence of non-users tested population to general measures of mask wearing and social distancing.

Intranasal administration of a composition in accordance with the present invention resulted in prevention of COVID-19 not only compared to occurrence in local population of Bnei Brak (almost 29%) or national (12.1%), but remarkably also compared to a population adhering to standard preventive measure as the population of the survey—no infection vs. 10% infection.

1. A composition in the form of dry powder for intranasal administration, said composition comprising first type particles, being essentially spherical solid particles comprising at least one physiologically acceptable mucoadhesive polymer and/or bioadhesive polymer and/or gel-forming polymer in combination with at least one functional additive, wherein at least about 90% of said first type particles are of size of about 10-300 microns, wherein at least 50% of the particles are of size of about 30-100 microns and less than about 10% of said first type particles are of size of about 5 microns.

2. The composition of claim 1, further comprising second type particles being irregularly shaped solid particles comprising at least one physiologically acceptable carrier, wherein said second type particles are of a mean particle size greater than that of the said first type particles, preferably a mean particle size of from about 50 to about 200 microns.

3. The composition of claim 1, wherein mucoadhesive polymer and said bioadhesive polymer is a hydrophilic or amphiphilic gel-forming polymer, said mucoadhesive polymer and or said bioadhesive polymer being any one of hydroxypropylmethyl cellulose (HPMC), hydroxypropyl cellulose (HPC), sodium carboxymethyl cellulose (CMC), a natural gum such a xanthan gum, guar gum, gum acacia and gum tragacanth, starch, chitosan, algal sulfated polysaccharides, hydrophilic methacrylic polymer, hydroxyethyl methacrylic polymer, hydrophilic acrylic acid polymer selected from Carbopol, polyethylene glycol, Poloxamer, polyvinyl alcohol and any co-polymer, grafted polymer or and any mixture of at least two thereof.

4. The composition of claim 1, wherein said mucoadhesive and/or said bioadhesive polymer and/or gel-forming polymer is hydroxypropyl-methyl cellulose (HPMC).

5. The composition of claim 1, wherein said at least one functional additive is a physiologically acceptable pH modifying agent, being an acidifying agent which following intranasal administration and dissolution of the particles, provides the nasal cavity environment with a pH equal to or lower than about 3.5.

6. The composition of claim 1, wherein said at least one functional additive is a physiologically acceptable acidifying agent being any one of L-pyroglutamic acid (PCA); ascorbic acid, citric acid, phytic acid, succinic acid, acetic acid, citric acid, hydrochloric acid, lactic acid, tartaric acid, malic acid, salts thereof hydrates, such as mono-, di- or tri-hydrates and anhydrates or monohydrate thereof.

7. The composition of claim 1, wherein said functional additive is at least one physiologically acceptable antibacterial agent and/or at least one physiologically acceptable preserving agent, being any one of benzalkonium chloride, ADBAC, benzoic acid, chlorocresol, diazolidinyl urea, imidurea, edetic acid and its salts, potassium sorbate, sorbic acid and its salts, benzethonium chloride or docusate, and any mixture of at least two thereof.

8. The composition of claim 1, wherein said functional additive comprises a mixture of at least one pH modifying agent, and at least one physiologically acceptable antibacterial and/or preserving agent.

9. The composition of claim 8, wherein said at least one pH modifying agent is a mixture of citric acid and sodium citrate and said physiologically acceptable biocidal/bactericidal agent is at least one benzalkonium chloride.

10. The composition of claim 1, wherein the ratio between said at least one bioadhesive polymer and said at least one functional additive in said first type particles is between about 90.0-99.99% w/w.

11. The composition of claim 1, further comprising at least one pharmaceutical, alimentary or cosmetic scenting agent in said first type particles and/or in said powder and/or in said second type particles.

12. The composition of claim 1, wherein following intranasal administration to a subject and dissolution within the nasal cavity of the subject, said composition forms a protective film layer adhered to or a gel layer deposited on the nasal mucosa of said subject, which film layer or gel layer prevents at least in part foreign particulate bodies from accessing the nasal epithelium or penetrating the body of the subject, wherein said particulate bodies are viral pathogens or allergens.

13. The composition of claim 12, wherein viral pathogen is a virus of any one of the Coronaviridae including SARS-CoV-2, Severe Acute Respiratory Syndrome virus (SARS-CoV) and Middle East Respiratory Syndrome (MERS), Orthomyxoviridae, such as any of Influenza virus type A, Influenza virus type B or Influenza virus type C or any subtype or reassortant thereof including swine Influenza type A virus subtype H1N1 and avian Influenza type A virus subtype H5N1, Filoviridae, such as Marburg virus (MARV) and Ebola virus (EBOV), Flaviviridae such as Zika virus (ZIKV), West Nile virus (WNV), Dengue virus (DENV) and Yellow Fever virus (YFV) or Poxviridae families, and sub-families thereof.

14. A composition in the form of dry powder for intranasal administration to a subject in need, said composition comprising essentially spherical solid particles comprising HPMC in combination with a physiologically acceptable pH modifying agent being an acidifying agent, and/or a physiologically acceptable antibacterial and or preserving agent, wherein at least about 90% of said particles are of a mean particle size of about 40-150 microns, wherein at least 50% of the particles are of a mean particle size of about 15-100 microns and less than about 10% of said particles are of a mean particle size of about 5-30, providing a metered effective nominal dose of said HPMC, wherein following intranasal administration to said subject and dissolution in the nasal cavity of said subject, said composition forms a protective polymeric film layer or gel layer on the nasal mucosa.

15. The composition of claim 14, wherein said a physiologically acceptable acidifying agent is any one of L-pyroglutamic acid (PCA); ascorbic acid, citric acid, phytic acid, succinic acid, acetic acid, citric acid, hydrochloric acid, lactic acid, tartaric acid, malic acid, salts thereof and hydrates and anhydrates thereof, and any mixture of at least two thereof.

16. The composition of claim 14, wherein physiologically acceptable antibacterial/preserving agent is at least one of any one of benzalkonium chloride, ADBAC, benzoic acid, chlorocresol, diazolidinyl urea, imidurea, edetic acid and its salts, potassium sorbate, sorbic acid and its salts, benzethonium chloride or docusate, and any mixture of at least two thereof.

17. The composition of claim 14, wherein said at least one functional additive comprises a mixture of a mixture of citric acid and sodium citrate with at least one benzalkonium chloride.

18. The composition of claim 14, wherein the ratio between said at least one mucoadhesive and/or bioadhesive polymer and said at least one functional additive in said particles is between about 90.0-99.99% w/w.

19. The composition of claim 14, wherein said protective polymeric film layer or gel layer on the nasal mucosa prevents at least in part foreign particulate bodies of a mean size of from about 0.3 to about 20 microns from accessing the nasal epithelium or penetrating the body of the subject, wherein said particulate bodies are viral pathogens or allergens, wherein said viral pathogen is a virus of any one of the Coronaviridae including SARS-CoV-2, Severe Acute Respiratory Syndrome virus (SARS-CoV) and Middle East Respiratory Syndrome (MERS), Orthomyxoviridae, such as any of Influenza virus type A, Influenza virus type B or Influenza virus type C or any subtype or reassortant thereof including swine Influenza type A virus subtype H1N1 and avian Influenza type A virus subtype H5N1, Filoviridae, such as Marburg virus (MARV) and Ebola virus (EBOV), Flaviviridae such as Zika virus (ZIKV), West Nile virus (WNV), Dengue virus (DENV) and Yellow Fever virus (YFV) or Poxviridae families, and sub-families thereof.

20. A method for preventing or reducing infection by a viral pathogen, particularly respiratory viral infection, comprising intranasally administering to a subject in need an effective amount of a composition as defined in claim 14, wherein administration is self-administration by said subject or by another person.

21. The method of claim 20, wherein said administration is repeated administration of from one time to six times daily, over a period of from 1 to 30 or more days.

22. The method of claim 20, for preventing or reducing acuteness of cytokine storm induced by said viral pathogen.

23. The method of claim 20, wherein said subject is also using protective social mask and/or adheres to social distancing.

24. The method of 20, wherein said effective dose of HPMC is from about 0.01 mg to 20 mg.

25. The method of 20, wherein said composition is administered from once to six times daily.

26. The method of claim 20, wherein said composition is contained in an airtight container or inhaler suitable for intranasal administration of a powder, wherein said container provides said composition at a metered dose and it suitable for multiple administrations, specifically wherein said container is of a volume of from about 10 ml to about 500 ml.
